(19) United States
(12) Patent Application Publication
(10) Pub. No.: US 2020/0005907 A1
(43) Pub. Date: Jan. 2, 2020

(54) Automatic Generation of User and Non-User Cohorts for The Estimation of Drug's Effect from Longitudinal Observational Data

(71) Applicants: International Business Machines Corporation, Armonk, NY (US); Teva Pharmaceutical Industries, Ltd., Petach-Tikva (IL)

(72) Inventors: Michal Ozery-Flato, Tel Aviv (IL); Chen Yanover, Zichron Yaakov (IL)

(21) Appl. No.: 16/022,228

(22) Filed: Jun. 28, 2018

(51) Int. Cl. G16H 10/20; G06N 5/04

(52) U.S. Cl. CPC G16H 10/20; G06N 5/04

(57) ABSTRACT

Embodiments of the present systems and methods may provide generation of user and non-user cohorts for the estimation of drug's effect from longitudinal observational data that provide reduction of biases, account for progression of disease over time, and improve statistical significance. For example, in an embodiment, a computer-implemented method for conducting an observational trial of a drug under study may comprise receiving data relating to a plurality of patients, the data including drug prescription information for each patient, assigning each of the plurality of patients to a trial cohort based on the drug prescription information, setting an index date for each of the plurality of patients, and conducting an observational drug trial using the generated cohorts and index dates.

### BACKGROUND

The present invention relates to techniques for generation of user and non-user cohorts for the estimation of drug's effect from longitudinal observational data.

Ideally, the effect of a treatment on a disease trajectory is assessed in randomized controlled trials (RCTs). A key phase in an RCT is the random assignment of participants to treatment and control groups, which minimizes potential biases between these groups. The treatment group receives the treatment under investigation, while the control group receives an alternative or a placebo treatment.

Alternatively, or in addition, the effect of a treatment on a disease outcome can be estimated from observational data using causal inference methods that emulate randomized control trials. Emulating an RCT with longitudinal observational data requires defining a cohort of users (i.e., the treatment group) and a corresponding cohort of non-users (i.e., the control group) for the studied drug, then comparing the disease outcome in these two cohorts within a certain follow-up period, while correcting for potential biases between these two cohorts. To that end, one needs to set, for each individual in the treatment and control groups, the point in time (termed index date) when the follow-up period begins.

A need arises for techniques that may generate user and non-user cohorts for the estimation of drug's effect from longitudinal observational data that reduce biases, account for progression of disease over time, and improve statistical significance.

### SUMMARY

Embodiments of the present systems and methods may provide generation of user and non-user cohorts for the estimation of drug's effect from longitudinal observational data that provide reduction of estimation biases, account for progression of disease over time, and improve statistical significance. The index date for users may be readily defined, for example, as the date of the first prescription of the studied drug. In embodiments, a corresponding date for non-users may be set based on a randomly selected drug prescribed to them.

For example, in an embodiment, a computer-implemented method for conducting an observational trial of a drug under study may comprise receiving data relating to a plurality of patients, the data including drug prescription information for each patient, assigning each of the plurality of patients to a trial cohort based on the drug prescription information, setting an index date for each of the plurality of patients, and conducting an observational drug trial using the generated cohorts and index dates.

In embodiments, the drug prescription information may comprise information relating to use of the drug under study for at least some of the plurality of patients and the drug prescription information may comprise information relating to use of at least one drug that is not under study for at least some of the plurality of patients. Assigning patients to cohorts may comprise assigning to a user cohort those patients that are using the drug under study and assigning to a non-user cohort those patients that are not using the drug under study. Setting the index date may comprise setting the index date for each patient in the user cohort to a date of first prescription of the drug under study and setting the index date for each patient in the non-user cohort to a date of first prescription of a drug that is not under study. Assigning patients to cohorts may further comprise when there are more patients using the drug under study than not using the drug under study, assigning to a user cohort a random subset of those patients that are using the drug under study and assigning to a non-user cohort those patients that are not using the drug under study and the patients that are using the drug under study that were not assigned to the user cohort, except those patients that used the drug under study before their index date and those patients that used the drug under study in a period after their index date. At least some of the patients in the non-user cohort may have index dates assigned based on dates of first prescription of different drugs that are not under study. The method may further comprise applying additional exclusion criteria to the user cohort and the non-user cohort.

In an embodiment, a system for conducting an observational trial of a drug under study may comprise a processor, memory accessible by the processor, and computer program instructions stored in the memory and executable by the processor to perform receiving data relating to a plurality of patients, the data including drug prescription information for each patient, assigning each of the plurality of patients to a trial cohort based on the drug prescription information, setting an index date for each of the plurality of patients, and conducting an observational drug trial using the generated cohorts and index dates.

In an embodiment, a computer program product for conducting an observational trial of a drug under study may comprise a non-transitory computer readable storage having program instructions embodied therewith, the program instructions executable by a computer, to cause the computer to perform a method comprising receiving data relating to a plurality of patients, the data including drug prescription information for each patient, assigning each of the plurality of patients to a trial cohort based on the drug prescription information, setting an index date for each of the plurality of patients, and conducting an observational drug trial using the generated cohorts and index dates.

### BRIEF DESCRIPTION OF THE DRAWINGS

The details of the present invention, both as to its structure and operation, can best be understood by referring to the accompanying drawings, in which like reference numbers and designations refer to like elements.

FIG. 1 illustrates an exemplary prescription history.

FIG. 2 is an exemplary data flow diagram of a process according to embodiments of the present systems and methods.

FIG. 3a illustrates an exemplary prescription history for a studied drug.

FIG. 3b illustrates an exemplary prescription history for a studied drug.

FIG. 4 is an exemplary block diagram of a computer system in which processes involved in the embodiments described herein may be implemented.

### DETAILED DESCRIPTION

Embodiments of the present systems and methods may provide generation of user and non-user cohorts for the estimation of drug's effect from longitudinal observational data that provide reduction of biases, account for progression of disease over time, and improve statistical significance.

The effect of an intervention on a disease trajectory may be estimated from observational data using causal inference methods that emulate randomized control trials (RCTs). A key phase in an RCT is random assignment of participants to intervention groups (e.g., drug A vs. drug B users), which minimizes potential biases between the groups. Correspondingly, emulating an RCT with longitudinal observational data requires setting, for each individual, the point in time (termed index date) when the intervention, or its alternative, has occurred.

The present methods and systems may address the task of estimating the effect of a drug from longitudinal observational data, when the alternative intervention is not using the drug and, consequently, there is no clear index-date for non-users of the drug. In RCTs, patients in the non-user (or control) group are usually given an alternative drug, for example, a placebo, to control for potential effects of offering a treatment, such as psychosomatic effects. In embodiments, the present methods and systems may set the index-date of patients in the non-user cohort with respect to a randomly chosen drug prescribed to them, thus minimizing potential biases between the user and non-user cohorts.

In embodiments, the present methods and systems may estimate the effect of a studied drug on a disease outcome, compared to the alternative of not using the drug. To that end, a cohort of users and a corresponding cohort of non-users for the studied drug may be defined, then the disease outcome in these two cohorts within a certain follow-up period may be compared, while correcting for potential biases between these two cohorts.

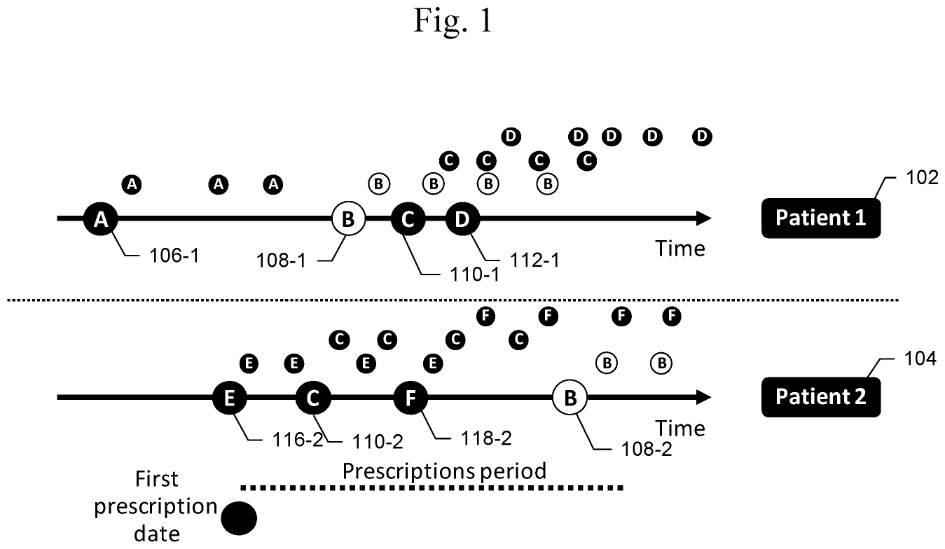

For example, for a study involving Parkinson's disease (PD) an estimate the effect of a drug on a certain disease outcome may be determined. For example, the study may estimate the extent to which the studied drug decreases (or increases) the prevalence of falls in PD patients. An exemplary illustrative prescription history is shown in FIG. 1 for two representative patients, Patient 1 102 and Patient 2 104, who are both diagnosed with PD. For simplicity of presentation, FIG. 1 shows just the prescription history of these two patients. Additional demographic and clinical information, such as diagnoses, appointments, lab tests, etc., is typically available and may be used to further refine the cohort definition and estimate the effect. In the example shown in FIG. 1, a first prescription for each drug A 106-1, B 108-1, 108-2, C 110-1, 110-2, D 112-1, E 116-2, and F 118-2 is shown for each patient 102, 104. Subsequent prescriptions for each drug are shown as well.

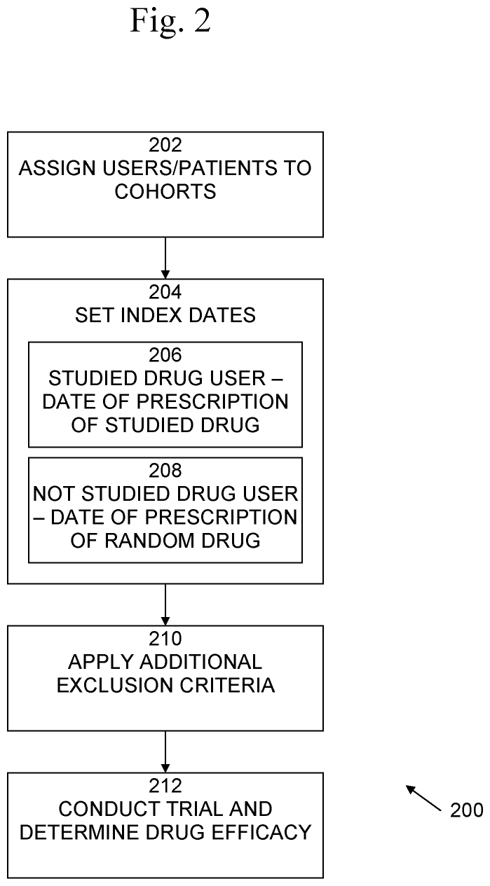

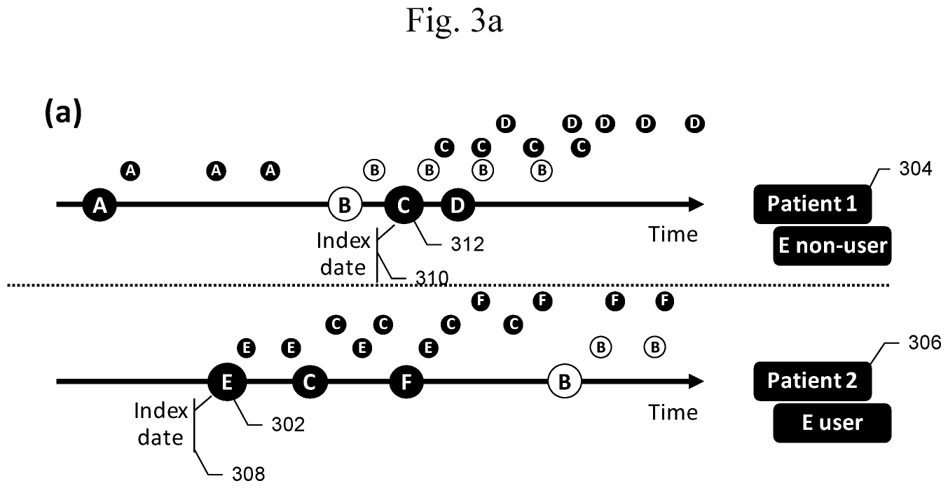

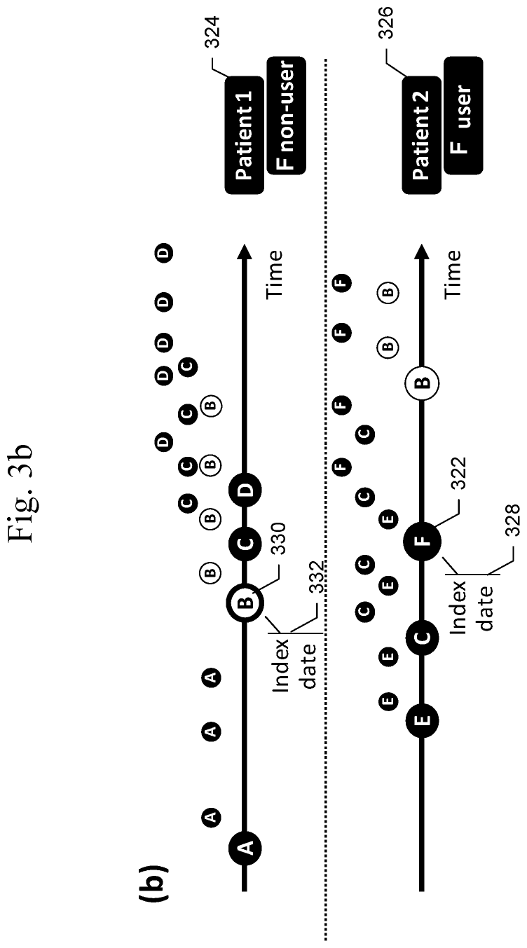

An example of a process 200 according to embodiments of the present systems and methods is shown in FIG. 2. It is best viewed in conjunction with FIGS. 3a and 3b, which illustrate the definitions of the user and non-user cohorts and the index dates. Process 200 begins with 202, in which patients who have been prescribed the studied drug may be assigned to the user cohort, and patients who have not been prescribed the studied drug may be assigned to the non-user cohort. For example, assuming that the studied drug is drug E 302, as shown in FIG. 3a, Patient 1 304 is not prescribed the studied drug and is assigned to the non-user cohort, while Patient 2 306 is prescribed the studied drug and is assigned to the user cohort.

At 204, the index-dates of the cohorts may be set. At 206, the index-date 308 may be set for each patient in the user cohort. For example, the index-date 308 may be set to the date of the first prescription of the studied drug for each patient 306 in the user cohort. At 208, the index-date 310 may be set for each patient in the non-user cohort. For example, for each patient 304 assigned to the non-user cohort, a drug prescribed to that patient, such as drug C 312, may be randomly selected, and the index-date 310 may be set to the date of the first prescription of that randomly-selected drug. It is to be noted that at least some of the patients in the non-user cohort may have index dates assigned based on dates of first prescription of different drugs that are not under study.

At 210, additional exclusion criteria may be applied to the two cohorts. Examples of such criteria may include requiring a minimal history before the index-date, or filtering out patients with or without certain events in their history. The non-user cohort may focus on patients who did not have a prescription to the studied drug; or also include patients whose prescription to the studied drug follows that of the random one (considered as censored at the date of the prescription of the studied drug).

In embodiments, in a case where there are more users than non-users of the drug under study, only a (random) subset of the patients that use the drug under study may be added to the user cohort, and the remaining patients may be added to the non-user cohort. In this case, patients in the non-user cohort will be excluded if they used the drug under study before the index-date, or may be considered censored if/when they start using the drug in the follow-up period after the index-date.

As another example, if the studied drug is drug F 322, as shown in FIG. 3b, then Patient 2 326 may be assigned to the drug F 322 user cohort, with the first prescription date used as the index date 328. Patient 1 324 may be assigned to the F non-user cohort, with drug B 330 randomly selected as the alternative drug for that patient, and its first prescription used as the index date 332.

Returning to FIG. 2, at 212, the observational drug trial may be conducted using the generated cohorts and index dates. During and/or upon completion of the observational drug trial the efficacy of the studied drug may be estimated based on the data gathered. Such efficacy may be estimated using statistical or other methods.

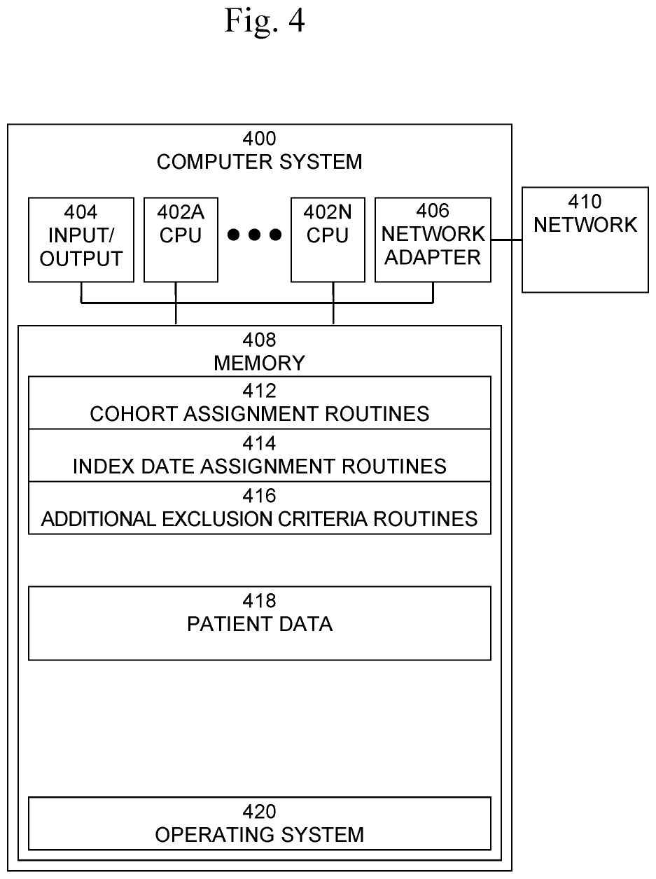

An exemplary block diagram of a computer system 402, in which processes involved in the embodiments described herein may be implemented, is shown in FIG. 4. Computer system 402 may be implemented using one or more programmed general-purpose computer systems, such as embedded processors, systems on a chip, personal computers, workstations, server systems, and minicomputers or mainframe computers, or in distributed, networked computing environments. Computer system 402 may include one or more processors (CPUs) 402A-402N, input/output circuitry 404, network adapter 406, and memory 408. CPUs 402A-402N execute program instructions in order to carry out the functions of the present communications systems and methods. Typically, CPUs 402A-402N are one or more microprocessors, such as an INTEL CORE® processor. FIG. 4 illustrates an embodiment in which computer system 402 is implemented as a single multi-processor computer system, in which multiple processors 402A-402N share system resources, such as memory 408, input/output circuitry 404, and network adapter 406. However, the present communications systems and methods also include embodiments in which computer system 402 is implemented as a plurality of networked computer systems, which may be single-processor computer systems, multi-processor computer systems, or a mix thereof.

Input/output circuitry 404 provides the capability to input data to, or output data from, computer system 402. For example, input/output circuitry may include input devices, such as keyboards, mice, touchpads, trackballs, scanners, analog to digital converters, etc., output devices, such as video adapters, monitors, printers, etc., and input/output devices, such as, modems, etc. Network adapter 406 interfaces device 400 with a network 410. Network 410 may be any public or proprietary LAN or WAN, including, but not limited to the Internet.

Memory 408 stores program instructions that are executed by, and data that are used and processed by, CPU 402 to perform the functions of computer system 402. Memory 408 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 408 may vary depending upon the function that computer system 402 is programmed to perform. In the example shown in FIG. 4, exemplary memory contents are shown representing routines and data for embodiments of the processes described above. However, one of skill in the art would recognize that these routines, along with the memory contents related to those routines, may not be included on one system or device, but rather may be distributed among a plurality of systems or devices, based on well-known engineering considerations. The present communications systems and methods may include any and all such arrangements.

In embodiments, at least a portion of the software shown in FIG. 4 may be implemented on a current leader server. Likewise, in embodiments, at least a portion of the software shown in FIG. 4 may be implemented on a computer system other than the current leader server.

In the example shown in FIG. 4, memory 408 may include cohort assignment routines 412, index date assignment routines 414, additional exclusion criteria routines 416, patient data 418, and operating system 420. Cohort assignment routines 412 may include software routines to perform assignment of patients to cohorts based on patient data 418. Index date assignment routines 414 may include software routines to perform assignment of index dates to patients based on patient data 418. Additional exclusion criteria routines 416 may include software routines to perform exclusion of patients to cohorts based on additional criteria included in patient data 418. Patient data 418 may include data, such as patient identification data, patient prescription data, patient medical history data, etc. Operating system 420 may provide overall system functionality.

As shown in FIG. 4, the present communications systems and methods may include implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A computer-implemented method for conducting an observational trial of a drug under study, the method comprising:
receiving data relating to a plurality of patients, the data including drug prescription information for each patient;
assigning each of the plurality of patients to a trial cohort based on the drug prescription information;
setting an index date for each of the plurality of patients; and
conducting an observational drug trial using the generated cohorts and index dates.

2. The method of claim 1, wherein the drug prescription information comprises information relating to use of the drug under study for at least some of the plurality of patients; and
the drug prescription information comprises information relating to use of at least one drug that is not under study for at least some of the plurality of patients.

3. The method of claim 2, wherein assigning patients to cohorts comprises:
assigning to a user cohort those patients that are using the drug under study; and
assigning to a non-user cohort those patients that are not using the drug under study.

4. The method of claim 3, wherein setting the index date comprises:
setting the index date for each patient in the user cohort to a date of first prescription of the drug under study; and
setting the index date for each patient in the non-user cohort to a date of first prescription of a drug that is not under study.

5. The method of claim 3, wherein assigning patients to cohorts further comprises:
when there are more patients using the drug under study than not using the drug under study, assigning to a user cohort a random subset of those patients that are using the drug under study; and
assigning to a non-user cohort those patients that are not using the drug under study and the patients that are using the drug under study that were not assigned to the user cohort, except those patients that used the drug under study before their index date and those patients that used the drug under study in a period after their index date.

6. The method of claim 4, wherein at least some of the patients in the non-user cohort have index dates assigned based on dates of first prescription of different drugs that are not under study.

7. The method of claim 5, further comprising applying additional exclusion criteria to the user cohort and the non-user cohort.

8. A system for conducting an observational trial of a drug under study, the system comprising a processor, memory accessible by the processor, and computer program instructions stored in the memory and executable by the processor to perform:
receiving data relating to a plurality of patients, the data including drug prescription information for each patient;
assigning each of the plurality of patients to a trial cohort based on the drug prescription information;
setting an index date for each of the plurality of patients; and
conducting an observational drug trial using the generated cohorts and index dates.

9. The system of claim 8, wherein the drug prescription information comprises information relating to use of the drug under study for at least some of the plurality of patients; and
the drug prescription information comprises information relating to use of a drug that is not under study for at least some of the plurality of patients.

10. The system of claim 9, wherein assigning patients to cohorts comprises:
assigning to a user cohort those patients that are using the drug under study; and
assigning to a non-user cohort those patients that are not using the drug under study.

11. The system of claim 10, wherein setting the index date comprises:
setting the index date for each patient in the user cohort to a date of first prescription of the drug under study; and
setting the index date for each patient in the non-user cohort to a date of first prescription of the drug that is not under study.

12. The system of claim 10, wherein assigning patients to cohorts further comprises:
when there are more patients using the drug under study than not using the drug under study, assigning to a user cohort a random subset of those patients that are using the drug under study; and
assigning to a non-user cohort those patients that are not using the drug under study and the patients that are using the drug under study that were not assigned to the user cohort, except those patients that used the drug under study before their index date and those patients that used the drug under study in a period after their index date.

13. The system of claim 11, wherein at least some of the patients in the non-user cohort have index dates assigned based on dates of first prescription of different drugs that are not under study.

14. The system of claim 12, further comprising applying additional exclusion criteria to the user cohort and the non-user cohort.

15. A computer program product for conducting an observational trial of a drug under study, the computer program product comprising a non-transitory computer readable storage having program instructions embodied therewith, the program instructions executable by a computer, to cause the computer to perform a method comprising:
receiving data relating to a plurality of patients, the data including drug prescription information for each patient;
assigning each of the plurality of patients to a trial cohort based on the drug prescription information;
setting an index date for each of the plurality of patients; and
conducting an observational drug trial using the generated cohorts and index dates.

16. The computer program product of claim 15, wherein the drug prescription information comprises information relating to use of the drug under study for at least some of the plurality of patients; and
the drug prescription information comprises information relating to use of a drug that is not under study for at least some of the plurality of patients.

17. The computer program product of claim 16, wherein assigning patients to cohorts comprises:
assigning to a user cohort those patients that are using the drug under study; and
assigning to a non-user cohort those patients that are not using the drug under study.

18. The computer program product of claim 17, wherein setting the index date comprises:
setting the index date for each patient in the user cohort to a date of first prescription of the drug under study; and
setting the index date for each patient in the non-user cohort to a date of first prescription of the drug that is not under study.

19. The computer program product of claim 18, wherein assigning patients to cohorts further comprises:
when there are more patients using the drug under study than not using the drug under study, assigning to a user cohort a random subset of those patients that are using the drug under study; and
assigning to a non-user cohort those patients that are not using the drug under study and the patients that are using the drug under study that were not assigned to the user cohort, except those patients that used the drug under study before their index date and those patients that used the drug under study in a period after their index date.

20. The computer program product of claim 18, wherein at least some of the patients in the non-user cohort have index dates assigned based on dates of first prescription of different drugs that are not under study.
